(19) United States
(12) Patent Application Publication
(10) Pub. No.: US 2012/0016251 A1
(43) Pub. Date: Jan. 19, 2012

(54) System for Respiration Data Processing and Characterization

(75) Inventors: Hongxuan Zhang, Palatine, IL (US); Detlef W. Koertge, Carpentersville, IL (US); Dennis Steibel, JR., Lake Zurich, IL (US); Harold James Wade, Rockford, IL (US)

(73) Assignee: SIEMENS MEDICAL SOLUTIONS USA, INC., Malvern, PA (US)

(21) Appl. No.: 13/042,806

(22) Filed: Mar. 8, 2011

Related U.S. Application Data
(60) Provisional application No. 61/364,429, filed on Jul. 15, 2010.

(51) Int. Cl. A61B 5/08

(52) U.S. Cl. 600/532

(57) ABSTRACT

A system for respiration or cardiac condition characterization and abnormality detection includes an interface that receives data representing a signal indicating concentration of carbon dioxide in patient gases over multiple signal cycles. A signal processor uses the received data in determining multiple amplitude related characteristic values. A comparator compares at least one of the amplitude related characteristic values or a value derived from the amplitude related characteristic values, with a threshold value to provide a comparison indicator. A patient monitor in response to the comparison indicator indicating an amplitude related characteristic value or a value derived from the amplitude related characteristic values, exceeds the threshold value, generates an alert message associated with the threshold.

This is a non-provisional application of provisional application Ser. No. 61/364,429 filed Jul. 15, 2010, by H. Zhang et al.

### FIELD OF THE INVENTION

This invention concerns a system for respiration or cardiac condition characterization and abnormality detection by determining multiple amplitude related characteristic values of a signal indicating concentration of carbon dioxide in patient gases over multiple signal cycles.

### BACKGROUND OF THE INVENTION

Respiration can be utilized to track and diagnose patient health status, especially of air pathways (directly) and blood flow (indirectly) due to the O2 consumption. The relationships between a respiration signal and patient pathologies can qualitatively and quantitatively be detected and characterized by calculating and analyzing parameters of the respiration signal. The determination of these relationships is of value for use in applying anesthesia and characterizing asthma, insufficient blood flow in heart coronaries, O2 consumption in coronary arteries and brain trauma injury. A cardiac respiration signal is a vital sign signal used to diagnose and characterize patient health status, especially in a situation where patient signals, such as ICEG (intra-cardiac electrogram) signals, ECG (electrocardiogram) signals, NIBP (non-invasive blood pressure) blood pressures, are noisy and not reliable.

Capnography systems, such as for end-tidal CO₂ (EtCO2) waveform shape analysis, are used in respiration signal analysis and quantification. However known waveform shape analysis methods do not provide quantitative waveform pattern analysis for early detection and characterization of emergency patient events and cardiac arrhythmia severity, including for asthma and myocardial ischemia. Further known patient respiration and capnograph analysis focuses on waveform amplitude (e.g., mmHg in an End-tidal CO2 waveform) and may be unable to capture small changes in a capnograph waveform, such as partial waveform changes, timing changes and especially waveform shape and timing changes due to pathologies.

Respiration and capnograph analysis in known patient monitoring and diagnosis systems focus on display of a waveform and key parameters of the signals, such as respiration rate, End-tidal CO2 value. The respiration signals and capnograph waveform analysis and trend detection are typically subjective and need extensive experience to interpret. Further known patient respiration signal and waveform analysis and monitoring are typically used independently and fail to provide a sensitive and reliable patient health status evaluation and diagnosis. A system according to invention principles addresses these deficiencies and related problems.

### SUMMARY OF THE INVENTION

A system characterizes respiration capnograph waveforms and patterns (including external respiration signals and internal blood oximetric signals, such as an SPO2 signal) to identify patient respiration abnormality and characterize patient health status, such as asthma level, myocardial ischemia and infarction and possibility of a stroke event. A system for respiration or cardiac condition characterization and abnormality detection includes an interface, a signal processor, a comparator and a patient monitor. The interface receives data representing a signal indicating concentration of carbon dioxide in patient gases over multiple signal cycles. The signal processor uses the received data in determining multiple amplitude related characteristic values comprising at least two of, (a) a magnitude of an amplitude of a baseline of the signal, (b) a magnitude of an amplitude at an end of early exhalation of the signal, (c) a magnitude of an amplitude of a start of the exhalation plateau of the signal, (d) a magnitude of an amplitude of an end tidal point of the signal and (e) a magnitude of an amplitude at an end of inhalation point of the signal. The comparator compares at least one of the amplitude related characteristic values or a value derived from the amplitude related characteristic values, with a threshold value to provide a comparison indicator. The patient monitor in response to the comparison indicator indicating an amplitude related characteristic value or a value derived from the amplitude related characteristic values, exceeds the threshold value, generates an alert message associated with the threshold.

### BRIEF DESCRIPTION OF THE DRAWING

FIG. 1 shows a system for respiration or cardiac condition characterization and abnormality detection that analyzes respiration and capnograph data and automatically controls treatment in response to respiration pattern analysis, e.g. by controlling heart rate via stimulation pulses and oxygen, according to invention principles.

FIG. 2 shows respiration and capnograph (CO2) signal waveforms.

FIG. 3 illustrates parameters of a capnograph waveform, according to invention principles.

FIG. 4 shows a table identifying respiration and CO2 parameters, according to invention principles.

FIG. 5 shows a table identifying ratios of respiration and CO2 parameters, according to invention principles.

FIG. 6 illustrates frequency domain analysis of different episodes of respiratory behavior, according to invention principles.

FIG. 7 shows an artificial neural network (ANN) used for respiration or cardiac condition characterization and abnormality detection, according to invention principles.

FIG. 8 shows a flowchart of a process performed by the system for respiration or cardiac condition characterization and abnormality detection, according to invention principles.

FIG. 9 shows an analysis of different capnograph episodes, according to invention principles.

FIG. 10 shows an implantable cardioverter-defibrillator (ICD), according to invention principles.

FIG. 11 shows a flowchart of a process used by a system for respiration or cardiac condition characterization and abnormality detection, according to invention principles.

### DETAILED DESCRIPTION OF THE INVENTION

A system improves accuracy and reliability of analysis and interpretation of respiration activities, by characterizing respiration capnograph waveforms and patterns (including external respiration signals and internal blood oximetric signals, such as SPO2 signals) to identify patient respiration abnormality and characterize patient health status, such as asthma level, myocardial ischemia and infarction and possibility of a stroke event. The system quantifies and determines statistical variation and variability of different portions of a respiration waveform and signals to provide a more precise time, type and severity of cardiac pathology and events for improved diagnosis, such as of cardiac arrhythmias. System statistical pattern analysis involves capnograph and ECG, ICEG, temperature and blood pressure signals, to diagnose and evaluate patient condition for real time patient monitoring and diagnosis.

The system analyzes cardiac respiration and capnograph data and determines patient health status to quantify a time, type and severity of cardiac pathology and events for improved diagnosis, such as of asthma and cardiac arrhythmias. The system performs multi-channel patient signal and data synchronization based capnograph pattern diagnosis and evaluation. A respiration signal is a vital sign signal used to monitor and diagnose health status of patients. The system employs variability and variation calculation and evaluation of respiration signals and data, especially of respiration waveforms (including End-tidal CO2 and CO2), to characterize and quantify oxygen consumption rate and respiration efficiency. The respiration and capnography analysis system is used for different kinds of oximetric signals, such as SPO2 to support an SPO2 sensor based intra-cardiac catheter system and cardiac event detection for ICD devices. The system identifies patient disorders, differentiates abnormalities, characterizes pathological severity, predicts life-threatening events and evaluates drug delivery effects.

Capnography is used for monitoring of the concentration or partial pressure of carbon dioxide (CO₂) in respiratory gases for use during anesthesia and intensive care. It is usually presented as a graph of expiratory CO₂ plotted against time, or expired volume. Capnography indicates how much CO2 is being eliminated from the lungs by measuring exhaled CO2 with a device that senses the CO2 level. It is a sensitive indicator of lung function and guides adjustment of a breathing machine or it may provide an early warning that lungs are not functioning properly. Capnography is also used for safety determination because it is a fast and reliable indicator of proper placement of a breathing (endrotracheal) tube, which is a tube extending from the nose or mouth into the windpipe.

The system captures variation of respiration and oximetric signals and quantitatively identifies cardiac pathologies. Respiration and oximetric signals include both non-invasive and invasive oximetric signals used in measurement and data acquisition, such as in respiration air monitoring, and include SPO2 signals and intra-cardiac O2 and CO2 signals. Nose and mouth respiration air monitoring is used for air pathway monitoring and health status evaluation for an entire patient body. Intra-cardiac O2 or CO2 content monitoring (e.g. by using catheter based transducers, sensors and detectors) is used for local organ and function diagnosis, such as of coronary artery function and arrhythmia detection, especially in ICD products. The system provides multi-channel signal based cardiac arrhythmia diagnosis and evaluation (such as by using an artificial neural network (ANN)) for use in an ICD and a catheter for intra-cardiac oximetric signal monitoring.

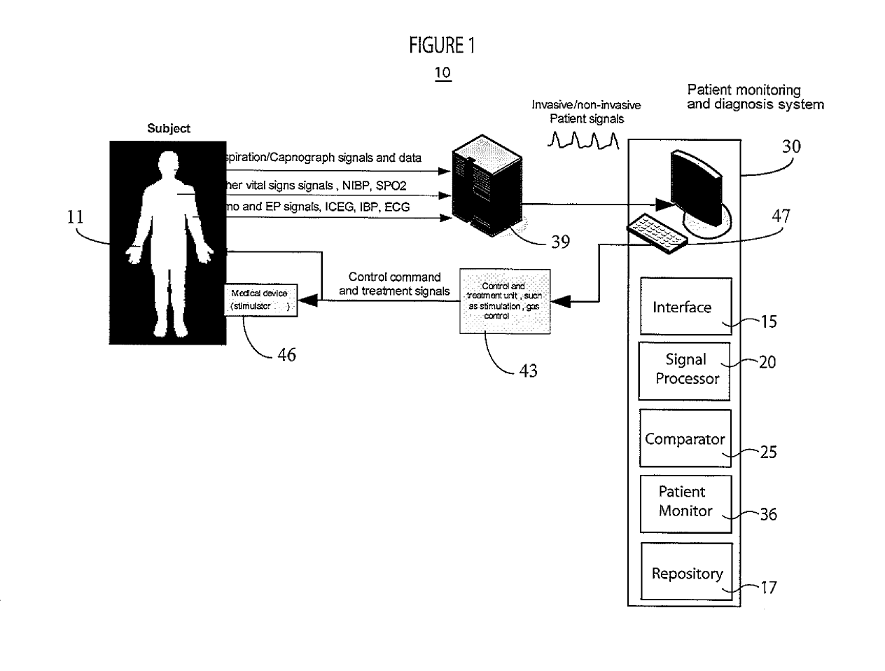

FIG. 1 shows system 10 for respiration or cardiac condition characterization and abnormality detection that analyzes respiration and capnograph data and automatically controls treatment in response to respiration pattern analysis, e.g. by controlling heart rate via stimulation pulses and oxygen. System 10 employs automatic control and treatment of patient pathology based on respiration mode and pattern analysis, e.g. by controlling the heart rate of a cardiac stimulator pulse and oxygen in the respiration. System 10 comprises a closed loop system for patient status and health monitoring and diagnosis including signal interface 39 and at least one computer system, workstation, server or other processing device 30 including interface 15, repository 17, patient monitor 36, signal processor 20, comparator 25 and a user interface 47. System 10 further includes control unit 43 for controlling treatment, electrical stimulation e.g. for heart pacing or ablation, respiration and anesthesia in response to parameters and calculation performed by processor 20. Medical device unit 46 provides treatment signals to patient 11 in response to treatment related signals provided by unit 43. Signal interface 39 receives and processes signals from patient 11, including respiration, vital signs, hemodynamic and electrophysiological (EP) signals that are acquired by different sensors and transducers in unit 39. The acquired signals are digitized and transmitted by unit 39 for analysis in computer system 30. In response to evaluation and diagnosis of multi-channel signals from patient 11, system 30 (or a user) determines appropriate control and treatment for the patient and the respiration and capnography data is synchronized with other signals from a patient.

Interface 15 receives data representing a signal indicating concentration of carbon dioxide in patient gases over multiple signal cycles. Signal processor 20 uses the received data in determining multiple amplitude related characteristic values comprising at least two of (a) a magnitude of an amplitude of a baseline of the signal, (b) a magnitude of an amplitude at an end of early exhalation of the signal, (c) a magnitude of an amplitude of a start of the exhalation plateau of the signal, (d) a magnitude of an amplitude of an end tidal point of the signal and (e) a magnitude of an amplitude at an end of inhalation point of the signal. Comparator 25 compares at least one of the amplitude related characteristic values or a value derived from the amplitude related characteristic values, with a threshold value to provide a comparison indicator. Patient monitor 36, in response to the comparison indicator indicating an amplitude related characteristic value or a value derived from the amplitude related characteristic values, exceeds the threshold value, generates an alert message associated with the threshold.

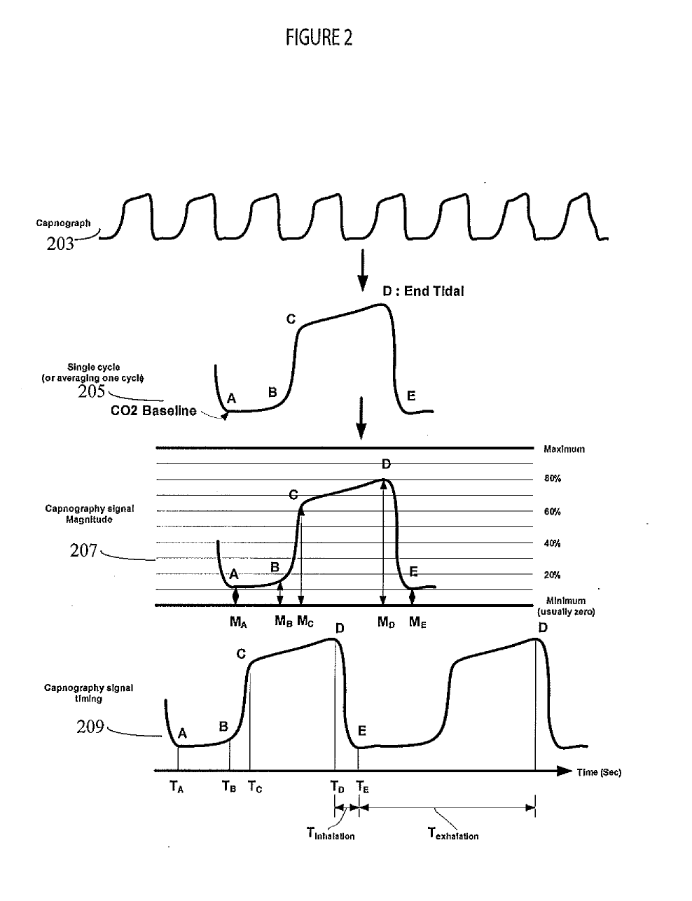

FIG. 2 shows respiration and capnograph (CO2) signal waveforms indicating waveform amplitude and time parameters used for further calculation and analysis involving inhalation and exhalation related calculation. System 10 (FIG. 1) processes CO2 signals and associated data (waveform, shape, magnitude, timing, phase) as well as oximetric signals, such as SPO2 signals, intra-cardiac O2/CO2 signals and blood pressure signals. FIG. 2 illustrates CO2 signal 203 (capnograph) respiration cycle points A, B, C, D, E in waveforms 205, 207 and 209. Amplitude and timing parameters are indicated that are used to track and calculate capnography waveform changes and variation, for example Tᵢₙₕₐₗₐₜᵢₒₙ and Tₑₓₕₐₗₐₜᵢₒₙ represent the time duration and length of inhalation and exhalation procedures, respectively.

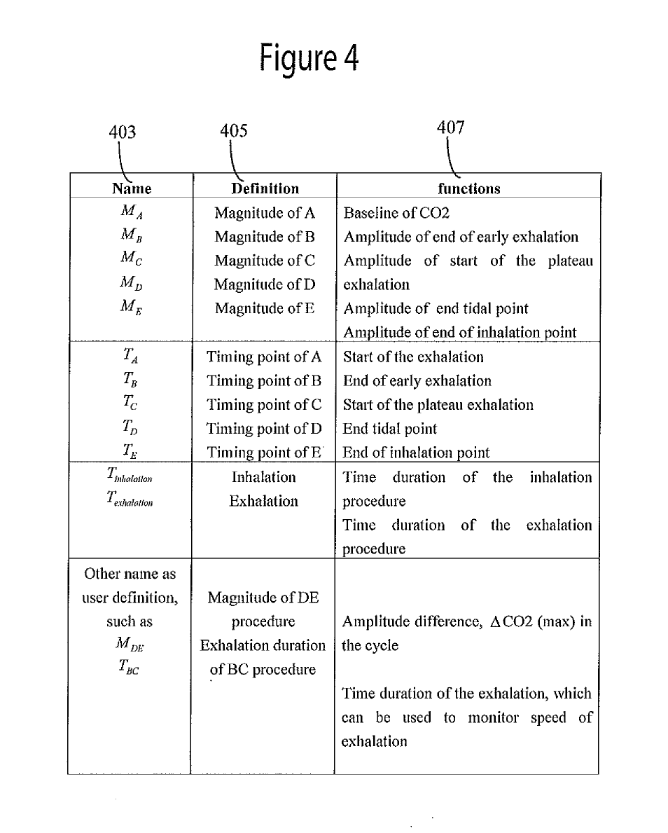

FIG. 4 shows a table identifying respiration and CO2 parameters associated with respiration cycle points A, B, C, D, E of FIG. 2. Specifically, the table identifies by name amplitude (M) and time duration (T) parameters in column 405 having identifiers in column 403 and described in column 407. The points A, B, C, D, E determine different time and function points in a capnograph waveform; for example, A to B is a baseline phase and beginning of exhalation, B to C is an early exhalation phase, C to D is an alveolar plateau phase, D is an end tidal point (end of exhalation) and D to E is an inhalation phase. System 10 uses the parameters of the table of FIG. 4 in qualitatively and quantitatively diagnosing and characterizing respiratory signals including inhalation and exhalation force, O2 consumption rate and respiration rate (air speed of inhalation and exhalation).

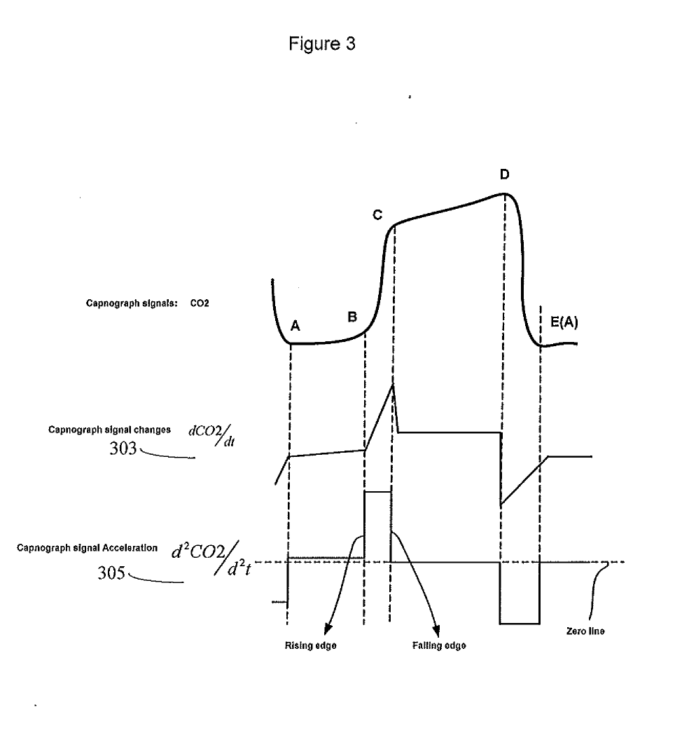

FIG. 3 illustrates parameters of a capnograph waveform in a complete respiration cycle indicating exhalation and inhalation procedures. A to B is post inspiration and dead space exhalation, B is the start of alveolar exhalation, B-C is the exhalation upstroke, C-D is a continuation of exhalation, or the plateau (the gas is alveolar, rich in CO2). D is the end-tidal value (peak CO2 concentration), D-E is the inspiration washout. System 10 performs respiration signal analysis using amplitude (e.g., peak) detectors and timing detectors (e.g., using clock counters) including threshold, minimum and maximum detectors and determines time points of A, B, C, D, E, based on rate of waveform change and signal amplitude detection. For example, D identifies a time of maximum amplitude (peak) in a capnography signal cycle; A coincides with time of minimum amplitude (waveform signal valley), B indicates time of alveolar exhalation at which the CO2 content change rate switches from slow to fast (as illustrated in waveform 303) and C indicates exhalation time point at which CO2 content rate of change switches from fast (high) to slow. The CO2 content rate change point is determined and detected by second order CO2 signal analysis,

d²CO2/d²t

as illustrated in waveform 305. Based on system 10 detection and determination of the different time points and amplitudes, the cycle of the capnograph waveform is segmented automatically and used in time and frequency domain analysis.

Signal processor 20 (FIG. 1) segments a capnograph signal waveform by determining an end tidal CO2 (EtCO2) maximum amplitude of the signal cycle using a peak detector and by determining a corresponding EtCO2 time point D in the respiration cycle (comprising the waveform between two EtCO2 peaks, for example). Processor 20 determines a first minimum amplitude after the EtCO2 peak at corresponding time point A indicating end of inhalation and beginning of exhalation using a minimum voltage detector. The CO2 content (density) value slowly increases from point A (A and E have the same time point and point A is determined in a sequence of successive respiration cycles). Processor 20 determines amplitude and corresponding time of point B which is a switching point from fast to slow exhalation and also determines amplitude and corresponding time of point C by calculating acceleration

d²CO2/d²t

in the capnograph waveform to detect a pulse falling edge in the CO2 acceleration signal (as illustrated in waveforms 303, 305 FIG. 3). Point C corresponds to a switching point from fast to slow exhalation. Processor 20 determines amplitude and corresponding time of point B by detecting a pulse rising edge (positive pulse) in the CO2 acceleration signal (as illustrated in waveforms 303, 305 FIG. 3). Signal processor 20 advantageously detects patient pathology using parameters of FIGS. 2-5 including different time and amplitude ratios.

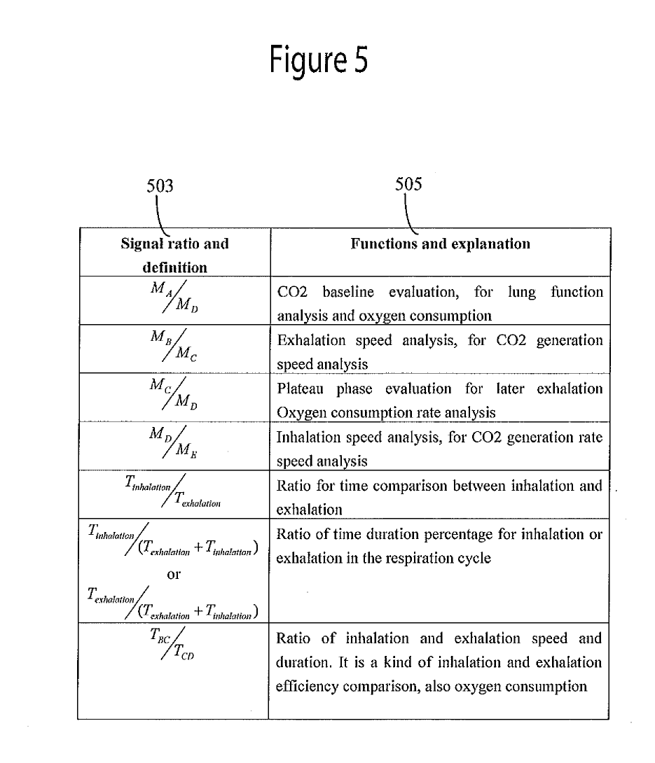

FIG. 5 shows a table identifying ratios of respiration and CO2 parameters calculated by signal processor 20 (FIG. 1). Specifically, column 503 identifies amplitude and time related ratio parameters of a capnograph waveform and column 505 provides a corresponding description of the parameters. Processor 20 further calculates variation and variability of the previously described parameters and ratios. Processor 20 employs the ratios and parameters of the tables of FIGS. 4 and 5 independently or in combination in detecting a patient medical condition. Additionally processor 20 employs different ratios such as T_{AB}/T_{CD}, M_{C}/M_{E} selected in response to user preference or data identifying a clinical application or procedure concerned. Processor 20 further calculates variation and variability as using the functions,

Mean or averaging value (expectation);

$$mean ( X ) = \frac{1}{N} {\sum }_{i ∈ N} X ( i ) ;$$

Standard deviation:

$$STD ( X ) = \frac{1}{N - 1} {\sum }_{i ∈ N - 1} ( X ( i ) - mean ( X ) )$$

$$Signal Variation = \frac{mean ( X )}{STD ( X )}$$

$$Signal Variability = \frac{max ( X - mean ( X ) )}{mean ( X )} .$$

where X is a timing or magnitude parameter or ratio of the tables of FIGS. 4 and 5 and N is the calculation window size (there are N respiration capnograph cycles in a shifting calculation window). The variation and variability analysis are used for air pathway characterization and evaluation and for cardiac pathology and event quantification, such as determination of severity, prediction of occurrence of a malfunction, determination of tissue location of an abnormality (especially where multi-channel oximetric signal analysis is performed by system 10).

In addition, during variation and variability analysis, processor 20 in one embodiment employs a low pass, high pass or band pass filter to eliminate noise due to a heart beat, patient movement or power line noise, for example.

System 10 (FIG. 1) employs respiration and capnograph analysis and calculations to track and characterize respiration activity. If capnograph data is coining from blood samples in different heart chambers, O2 consumption rate and cardiac pathology information is derived by the analysis. Processor 20 compares a current (real time) respiration signal with a previously determined baseline signal in analyzing a capnograph waveform by synchronizing different signals using an end tidal CO2 point of the different respiration cycle signals. Processor 20 determines mutual correspondence (MC) of two capnograph signals, as used herein, comprising a calculated value representative of similarity, difference or mutual information of corresponding sample points of the two signals over a set of sample points. The inventors have recognized that the application of information theory (including probability, entropy, and mutual information theory) based cardiac signal processing, which includes the application of mutual correspondence analysis to respiration and capnograph signals, produces an enhanced measurement value that is useful in analysis and diagnosis of patient conditions.

A respective cardiac electrophysiological signal is a random variable, with sampled values of the signal as events representing the outcomes of independent trials. It is possible to determine a probability distribution function for the signal values. The joint probability distribution of two signals S1 and S2 is the probability that both s1ᵢ=a and s2ᵢ=b for values a, b at each point i over sample points 0<i<N+1. If s1₁=a and s2ᵢ=b are independent events, their joint probability distribution is equal to the product of the probability of S1 and S2. The joint probability is

p(S1,S2)=p(S1)·p(S2|S1)

or the product of the probability of and the probability of s2ᵢ=b, given that s1ᵢ=a has occurred.

Entropy, or the information energy within a signal, is a measure of the uncertainty associated with a random variable. The entropy of a signal quantifies, in the sense of an expected value, the information contained in a message, usually in units such as bits. The entropy of two probability distributions P_{X} and P_{Y} is,

$$H ( XY ) = - {\sum }_{( x , y )} {p}_{xy} log ( {p}_{xy} )$$

Mutual information is the shared information between two signals and is the amount of information gained about X when Y is known, and vice versa. Mutual information is given by,

I(X,Y)=H(P_{X})+H(P_{Y})−H(P_{XY}).

When two signals contain zero mutual information, they represent independent random variables. Two identical signals have mutual information equal to 1. However, a signal and its mirror image also have mutual information equal to 1. Thus, a different measure to quantify signal similarity or difference is desirable for signal diagnostic interpretation. The value representing mutual correspondence calculated by a calculation processor represents pathology and cardiac malfunction related changes and is used to more sensitively characterize signal distortion and changes. Known information theory is employed using only a single parameter, e.g., amplitude. Given two processes, the corresponding output signals may be designated S₁ and S₂. One dimensional mutual correspondence of S₁ and S₂ may be determined by

$$MC ( {S}_{1} , {S}_{2} ) = \frac{{\sum }_{i = 1}^{N} {p}_{i} ( {s}_{1 i} , {s}_{2 i} )}{N}$$

where pᵢ is the joint probability of signal values s₁ᵢ and s₂ᵢ at sample point i, and the probability is summed over N sample points and normalized by dividing the sum by the value N.

Mutual correspondence (MC) is used by system 10 (FIG. 1) to track changes and variation of capnograph signals. System 10 (or a user) adaptively selects a baseline respiration cycle signal which can be used as a reference comparison signal (e.g., S1) to determine change in a patient monitored signal or to calculate a change range. Once the baseline (usually a benign) signal is determined, a real time capnography signal is captured (as an S2 signal) and used to calculate MC using the baseline signal, MC(S1, S2). System 10 compares a capnography signal with a baseline signal and derives real time mutual correspondence during continuous patient monitoring. In addition, the signals used in the MC calculation (S1, S2) can be single capnography cycle, a multiple cycle signal or an averaged cycle signal determined using a shifting time window over multiple cycles (for example, averaging over 5 respiration cycles). Changes and variation in respiration function due to pathologies or events can be small initially and buried in a signal. System 10 may employ different methods to extract and capture the small changes. System 10 frequency analysis processes capnograph data to determine shift in dominant frequency during an inhalation process and average frequency variation for an exhalation process. System 10 also determines a signal energy representative value for the different respiratory phases and an energy distribution pattern to track patient health status.

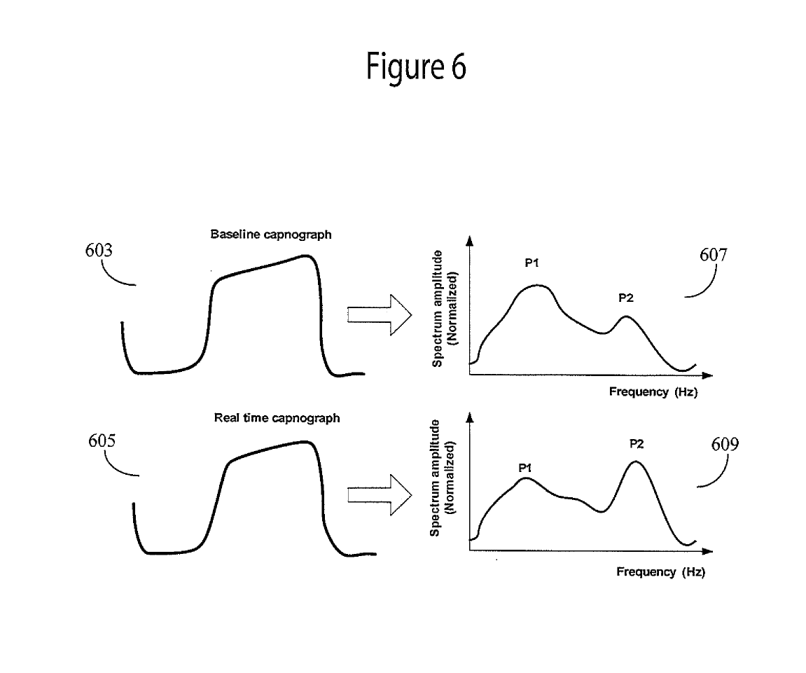

FIG. 6 illustrates frequency domain analysis of different episodes of respiratory behavior. The episodes are represented by a (normal) baseline waveform 603 and a real time respiration waveform 605 with respective corresponding frequency distributions 607 and 609. Frequency analysis of the capnograph waveform is sensitive to frequency change which may not be visible in the corresponding time domain signal amplitude waveform and end tidal CO2 amplitude value. Frequency distributions 607 and 609 show obvious frequency shifting, such as of a dominant frequency, and provide a sensitive quantitative analysis. System 10 calculates a dominant frequency ratio,

$${Ratio}_{Dominant \_ frequency} = \frac{{S}_{real} ( dominant\_frequency )}{{S}_{baseline} ( dominant\_frequency )}$$

In which, dominant frequency is the frequency of highest (peak) spectrum in the frequency domain distribution of the capnograph, for example P1 is the dominant frequency for the baseline waveform (603) while P2 is the dominant frequency in the real time episode waveform (605). System 10 determines a Dominant frequency shift parameter used to track pattern changes in a respiration signal.
System 10 calculates spectrum/energy ratio,

$${Ratio}_{Spectrum} = \frac{{\int }_{Ω} {\vert f ( real\_time ) \vert}^{2}}{{\int }_{Ω} {\vert f ( baseline ) \vert}^{2}}$$

In which,

$${\int }_{Ω} {\vert f ( \cdot ) \vert}^{2}$$

is a spectrum integration of a capnograph and respiration signal, Ω is a frequency bandwidth of interest, for example, 1 to 45 Hz.

System 10 may employ different frequency domain analysis methods to track capnograph signal changes in the frequency domain, including by averaging frequency spectrum values in a region of interest frequency bandwidth, such as 1 to 10 Hz. Further, system 10 determines different parameters and ratios in either time domain or frequency domain that reflect different characteristics of the respiration signals and capnograph data. In addition, the time and frequency domain calculated data is used in combination to improve analysis accuracy and sensitivity. Furthermore, the time frequency joint domain based analysis is used to extract valuable information identifying early changes due to patient pathologies and events.

System 10 employs multi-channel capnograph analysis to provide additional information about patient status and pathology events. For example, multi-channel (oximetric) signals are used by system 10 in comparison of oximetric data from different respiration sensors and transducers associated with different regions of the patient body (non-invasive, invasive, nose, mouth, blood vessel). The combined analysis advantageously identifies small variances to support identification of pathology location and severity. Further, the multi-channel signal calculations involve vital sign signals, hemodynamic signals and electrophysiological signals, such as SPO2, NIBP, ICEG, ECG and IBP signals. Different calculated results are combined to characterize clinical patient health status, especially of an air pathway and to detect cardiac arrhythmias. The system may use multi-channel analysis involving use of a Fuzzy system or expert system. System 10 in one embodiment uses ANN (artificial neural network) based comprehensive decision analysis for multi-channel and multi-parameter calculation based patient monitoring.

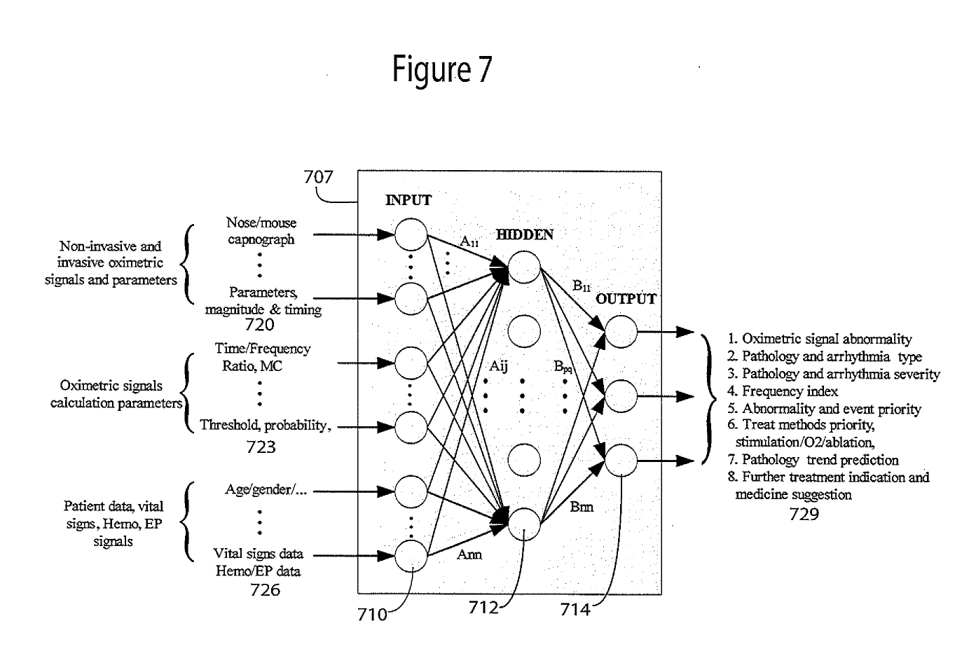

FIG. 7 shows an artificial neural network (ANN) used for respiration or cardiac condition characterization and abnormality detection. ANN unit 707 performs respiration and capnograph analysis for heart function characterization. ANN unit 707 processes an oximetric signal (such as respiration, SpO2) 720, calculated oximetric signal associated parameters 723 and patient vital sign data 726 in deriving output parameters 729. Output parameters 729 include data identifying oximetric signal abnormality, pathology and arrhythmia type, a pathology severity indicator, a frequency parameter, abnormality and event priority, treatment method, a pathology trend indication, a pathology type indication and candidate treatment suggestions. ANN unit 707 structure comprises 3 layers, an input layer 710, hidden layer 712 and output layer 714. ANN unit Aᵢⱼ weights are applied between input layer 710 and hidden layer 712 components of the ANN computation and B^{pq} weights are applied between hidden layer 712 and calculation index components 714 of the ANN computation. The Aᵢⱼ weights and B_{pq} weights are adaptively adjusted and tuned using a training data set. ANN unit 707 incorporates a self-learning function that processes signals 720, 723 and 726 to increase the accuracy of calculated results.

ANN unit 707 processes and maps input signals 720, 723 and 726 to a candidate diagnosis or treatment suggestion 729 to localize a tissue impairment within an organ and determine time of occurrence within a heart cycle. ANN unit 707 also identifies arrhythmia type (e.g., AF, MI, VT, VF), severity of arrhythmia treatment and urgency level and is usable for automatic heart condition detection, diagnosis, warning and treatment. Further unit 707 performs statistical analysis to construct a threshold used to detect tissue impairment and diagnose and predict cardiac arrhythmia and pathology.

Following a training phase with a training data set, ANN unit 707 maps signals 720, 723 and 726 to data 729. The system may be advantageously utilized in general patient monitoring and implantable cardiac devices for real time automatic analysis and detection of cardiac arrhythmias and abnormalities. ANN unit 707 is usable in multi-channel signal analysis and pattern analysis, for cross channel comparison and to further define arrhythmia type and location.

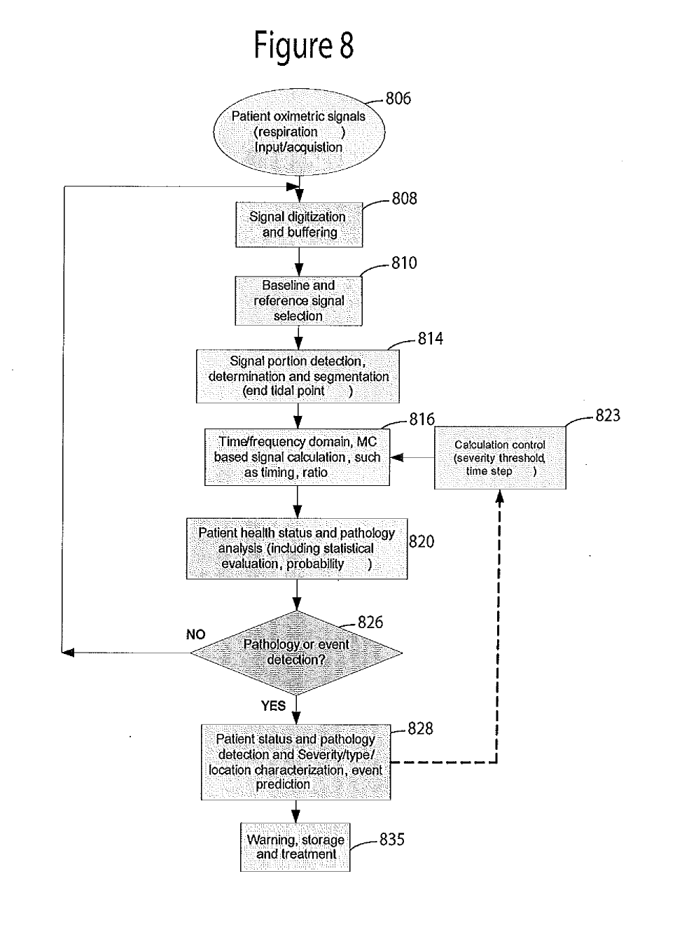

FIG. 8 shows a flowchart of a process performed by system 10 (FIG. 1) for respiration and capnograph signal analysis for cardiac condition characterization and abnormality detection. The respiration signal analysis is usable for characterization of anesthesia, asthma, brain injury and cardiac arrhythmia. Interface 15 in steps 806 and 808 acquires and digitizes respiration and capnograph signals and filters the acquired signals to reduce power line noise and patient movement noise. Signal processor 20 in step 810 selects a baseline reference signal for comparison with an acquired signal. In step 814, signal processor 20 performs signal pre-analysis by identifying a signal cycle and different segments and points such as end tidal point and exhalation starting point of a cycle of an acquired capnograph signal as shown in FIG. 2, for example.

In step 816, signal processor 20 determines and calculates the parameters of the tables of FIGS. 4 and 5 including the time domain and frequency domain signal ratios, variation, variability and mutual correspondence by comparison with the baseline reference signal. Signal processor 20 also performs a frequency domain analysis and statistical and probability evaluation in patient health status and pathology identification in step 820. Signal processor 20 in step 826 detects pathology and events in response to comparison of the determined and calculated parameters with predetermined parameter thresholds and ranges. The thresholds and ranges are user determinable or adaptively selected by processor 20. The process steps 808-826 are iteratively repeated until pathology or an event is detected in step 826. Signal processor 20 in step 828 employs mapping information in repository 17, associating ranges of the determined and calculated parameters with corresponding medical conditions in determining patient medical conditions, events and patient health status by comparing the determined and calculated parameters with the ranges to provide a comparison indicator identifying a medical condition. Processor 15 also determines the severity and location of the condition based on anatomical site of acquired source signal data. The comparison indicator triggers a warning and further CRM (cardiac rhythm management) intervention.

If signal processor 15 and comparator 20 in step 828 determine a medical condition indicating cardiac impairment or another abnormality is identified, patient monitor 19 in step 835 generates an alert message identifying the medical condition and abnormality and communicates the message to a user. Processor 15 in step 823 adaptively adjusts calculation time step, the selected portions and ROI of a filtered signal analyzed and adjusts a threshold employed by comparator 20 to improve medical condition detection. System 10 uses the oximetric signal and data quantification and characterization to monitor, diagnose and evaluate clinical asthma and anesthesia (based on external respiration CO2 signals) and myocardial ischemia and infarction (based on intra-cardiac CO2 and O2 signals).

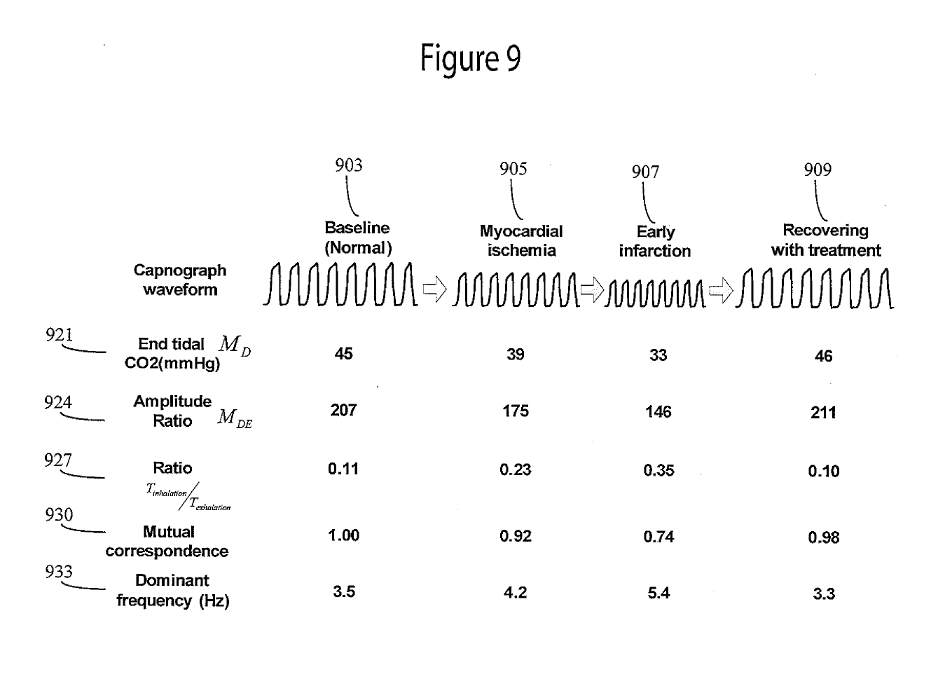

FIG. 9 shows a simulated case of myocardial ischemia (LAD occlusion) involving four episodes of capnograph signal changes. The four signal episodes are baseline 903, myocardial ischemia 905, early infarction 907, and recovery with treatment 909. The different capnograph signal episodes are compared using multiple different parameters and calculated parameters as illustrated in the tables of FIGS. 4 and 5. The parameters include end tidal CO2 921, amplitude ratio M_{DE} 924, time duration ratio Tᵢₙₕₐₗₐₜᵢₒₙ/Tₑₓₕₐₗₐₜᵢₒₙ 927, mutual correspondence 930, and dominant frequency 933 (the parameters calculated are selected by system 10 or a user). The different stages of patient condition comprise normal (baseline), low risk pathology (myocardial ischemia), high risk pathology (myocardial infarction), and recovery (with treatment). The parameters are determined for a selected respiration cycle, however the parameters may alternatively be determined for an averaged respiration cycle of several cycles within a shifting window (e.g., a 5 cycle window). In addition, processor 20 determines variation and variability to provide more capnograph analysis data.

In the (baseline) normal episode 903 the values for calculated end tidal CO2 value, magnitude ratio, time duration ratio, mutual correspondence and dominant frequency (highest frequency peak value) are 45, 207, 0.11, 1.00 and 3.5 respectively. In contrast, the corresponding calculated values of the early infarction episode 907 are 33, 146, 0.35, 0.74, and 5.4 (showing greater than 30% change compared with corresponding baseline values). Processor 20 determines from the change in parameter values (or via associated statistical tests and confidence level tests) that myocardial infarction is occurring and the calculated results are used by processor 20 to suggest medical treatment. After the treatment, the values of the 5 calculations go back to normal: 46, 211, 0.10, 0.98, and 3.3 (showing less than 5% change compared with corresponding baseline values).

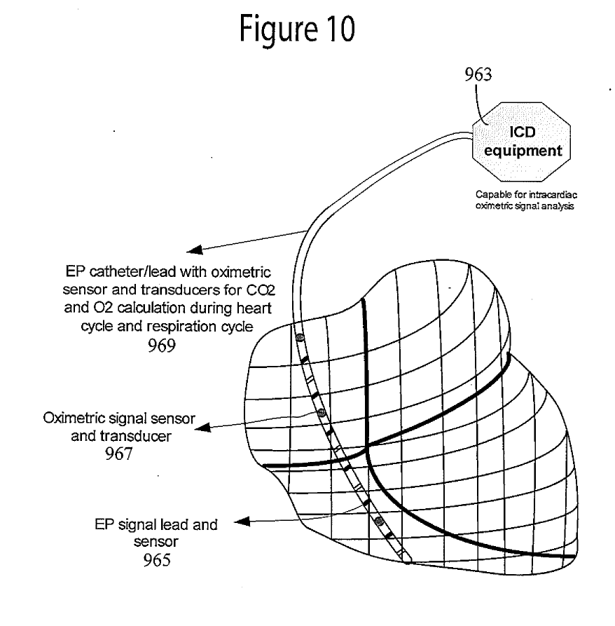

FIG. 10 shows an implantable cardioverter-defibrillator (ICD) 963 incorporating system 10 functions. Oximetric signals (such as external non-invasive respiration signals, intra-cardiac blood vessel CO2/O2 signals acquired by chemical sensors and optical sensors, invasively, non-invasively) are analyzed by processor 20 calculation of the parameters of the tables of FIGS. 4 and 5. The calculated parameters are used to characterize cardiac functions, for use in ICD device 963 for cardiac function monitoring and treatment, such as blood flow and coronary artery blockage monitoring. ICD system 963 performs oximetric signal (such as O2, CO2 data) analysis as well as intra-cardiac electrophysiological signal analysis. The ICD device uses multi-channel sensors and transducers, to capture real time signals, such as CO2 data, O2 data, EP signals, pressure signals.

Additionally system 10 multi-channel signal analysis is applied in 2-dimension and 3-dimension oximetric heart function mapping. System 10 multi-dimensional oximetric signal timing and parameter mapping distribution information is used in real time for cardiac function diagnosis and determining abnormal tissue location, potential abnormal pathways and arrhythmia severity in a cardiac diagram visual presentation prompting treatment. ICD device 963 comprises multi-channel sensors and transducers 965 and 967, which capture real time signals, including EP, oximetric, O2 and CO2 signals from multiple different anatomical sites acquired by multi-channel catheter 969 (or multiple different catheters), for example.

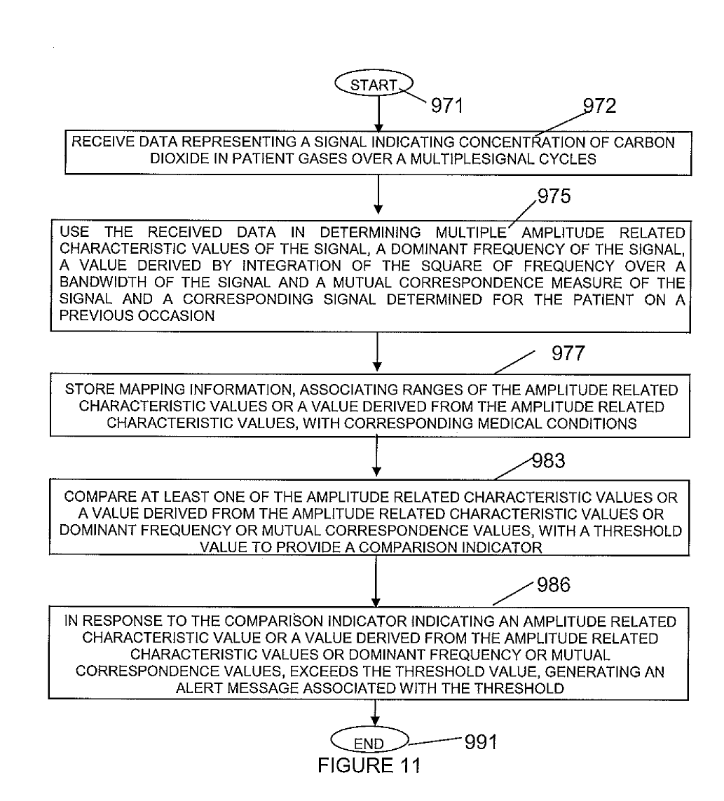

FIG. 11 shows a flowchart of a process used by system 10 for respiration or cardiac condition characterization and abnormality detection. In step 972 following the start at step 971, interface 15 receives data (e.g., sampled data) representing a signal indicating concentration of carbon dioxide in at least one of, (a) respiratory gases and (b) intra-cardiac gases over multiple signal cycles comprising respiratory cycles if respiratory carbon dioxide data is being processed and cardiac cycles if cardiac carbon dioxide data is being processed. Signal processor 20 in step 975 uses the received data in determining amplitude related characteristic values comprising (a) a magnitude of an amplitude of a baseline of the signal, (b) a magnitude of an amplitude at an end of early exhalation of the signal, (c) a magnitude of an amplitude of a start of the exhalation plateau of the signal, (d) a magnitude of an amplitude of an end tidal point of the signal and (e) a magnitude of an amplitude at an end of inhalation point of the signal.

Signal processor 20 employs a heart cycle synchronization signal in determining the amplitude related characteristic values and provides a value derived from the amplitude related characteristic values. In one embodiment processor 20 provides a value derived from the amplitude related characteristic values by averaging over multiple cycles and the value derived from the amplitude related characteristic values comprises a ratio of at least two of the amplitude related characteristic values. Processor 20 also provides a value derived from the amplitude related characteristic values by determining a ratio of an average to a standard deviation or variance of values and provides a value derived from the amplitude related characteristic values by determining a standard deviation or variance over multiple cycles. Signal processor 20 also uses the received data in determining a frequency related characteristic value comprising at least one of, a dominant frequency of the signal and a value derived by integration of the square of frequency over a bandwidth of the signal for comparison by comparator 25 of the frequency related characteristic value with a corresponding normal derived value. Processor 20 generates a ratio of the frequency related characteristic value to the corresponding normal derived value to compare values. Processor 20 further uses received sampled data in determining a mutual correspondence measure of the signal and a corresponding signal determined for the patient on a previous occasion. In one embodiment the mutual correspondence measure is derived using a function of the form,

$$MC ( {S}_{1} , {S}_{2} ) = \frac{{\sum }_{i = 1}^{N} {p}_{i} ( {s}_{1 i} , {s}_{2 i} )}{N}$$

where pᵢ is the joint probability of signal values s₁ᵢ and s₂ᵢ at sample point i, and the probability is summed over N sample points and normalized by dividing the sum by the value N. Processor 20 also determines a time associated with the different amplitude related characteristic values. Processor 20 further processes the received data in determining (i) a time duration of an inhalation process and (ii) a time duration of an exhalation process and calculates a ratio including the time duration of the inhalation process and the time duration of the exhalation process.

In step 977 processor 20 stores predetermined mapping information in repository 17. The mapping information associates predetermined thresholds and ranges of the amplitude related characteristic values, frequency related characteristic values and mutual correspondence measures and associates ranges of the characteristic values and measures (and values derived therefrom), with corresponding medical conditions. The predetermined mapping information associates ranges of the amplitude related characteristic values or a value derived from the amplitude related characteristic values and other characteristic values and measures, with particular patient demographic characteristics and with corresponding medical conditions and the system uses patient demographic data including at least one of, age weight, gender and height in comparing the amplitude related characteristic values or a value derived from the amplitude related characteristic values and other characteristic values and measures, with the ranges and generates an alert message indicating a potential medical condition

In step 983 comparator 25 compares at least one of the amplitude related characteristic values or a value derived from the amplitude related characteristic values and other characteristic values and measures, with a threshold value to provide a comparison indicator. Comparator 25 also compares the frequency related characteristic value with a corresponding normal derived value. Patient monitor 36 in step 986, in response to the comparison indicator indicating an amplitude related characteristic value or a value derived from the amplitude related characteristic values or other characteristic values and measures, exceeds the threshold value, and/or in response to frequency and mutual correspondence related comparisons, generates an alert message associated with the threshold. Patient monitor 36 also generates an alert message in response to determined ratios exceeding a predetermined threshold. The process of FIG. 11 terminates at step 991.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a computer, controller or microprocessor, for example, and is conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouth, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of FIGS. 1-11 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A respiration and capnography analysis system is used for different kinds of oximetric signals, such as SPO2 to support an SPO2 sensor based intra-cardiac catheter system and cardiac events detection for ICD devices to identify patient disorders, differentiate abnormalities, and characterize pathological severity. Further, the processes and applications may, in alternative embodiments, be located on one or more (e.g., distributed) processing devices on a network linking the units of FIG. 1. Any of the functions and steps provided in FIGS. 1-11 may be implemented in hardware, software or a combination of both.

What is claimed is:

1. A system for respiration or cardiac condition characterization and abnormality detection, comprising:
an interface for receiving data representing a signal indicating concentration of carbon dioxide in patient gases over a plurality of signal cycles;
a signal processor for using the received data in determining a plurality of amplitude related characteristic values comprising at least two of,
(a) a magnitude of an amplitude of a baseline of said signal,
(b) a magnitude of an amplitude at an end of early exhalation of said signal,
(c) a magnitude of an amplitude of a start of the exhalation plateau of said signal,
(d) a magnitude of an amplitude of an end tidal point of said signal and
(e) a magnitude of an amplitude at an end of inhalation point of said signal;
a comparator for comparing at least one of said amplitude related characteristic values or a value derived from said amplitude related characteristic values, with a threshold value to provide a comparison indicator; and
a patient monitor for in response to said comparison indicator indicating an amplitude related characteristic value or a value derived from said amplitude related characteristic values, exceeds the threshold value, generating an alert message associated with the threshold.

2. A system according to claim 1, wherein
said value derived from said amplitude related characteristic values comprises a ratio of at least two of said amplitude related characteristic values.

3. A system according to claim 1, wherein
said signal processor determines a time associated with at least two of said plurality of amplitude related characteristic values.

4. A system according to claim 1, wherein
said signal processor uses the received data in determining
(i) a time duration of an inhalation process and
(ii) a time duration of an exhalation process.

5. A system according to claim 1, wherein
said signal processor calculates a ratio including said time duration of said inhalation process and said time duration of said exhalation process.

6. A system according to claim 1, wherein
said signal indicates concentration of carbon dioxide in at least one of, (a) respiratory gases and (b) intra-cardiac gases.

7. A system according to claim 1, wherein
said signal processor employs a heart cycle synchronization signal in determining said amplitude related characteristic values.

8. A system according to claim 1, wherein
said signal processor provides said value derived from said amplitude related characteristic values by averaging over a plurality of cycles.

9. A system according to claim 1, wherein
said signal processor provides said value derived from said amplitude related characteristic values by determining a standard deviation or variance over a plurality of cycles.

10. A system according to claim 1, wherein
said signal processor provides said value derived from said amplitude related characteristic values by determining a ratio of an average to a standard deviation or variance of values.

11. A system according to claim 1, including
a repository of predetermined mapping information, associating ranges of said amplitude related characteristic values or a value derived from said amplitude related characteristic values, with corresponding medical conditions and
said comparator compares said amplitude related characteristic values or a value derived from said amplitude related characteristic values, with said ranges to provide a comparison indicator identifying a medical condition and
said patient monitor generates an alert message identifying said medical condition.

12. A system according to claim 11, wherein
said predetermined mapping information associates ranges of said amplitude related characteristic values or a value derived from said amplitude related characteristic values, with particular patient demographic characteristics and with corresponding medical conditions and said system uses patient demographic data including at least one of, age weight, gender and height in comparing said amplitude related characteristic values or a value derived from said amplitude related characteristic values, with said ranges and generating an alert message indicating a potential medical condition.

13. A system according to claim 1, wherein
said signal cycles are (a) respiratory cycles if respiratory carbon dioxide data is being processed and (b) cardiac cycles if cardiac carbon dioxide data is being processed.

14. A system according to claim 1, wherein
said received data comprises received sampled data.

15. A system for respiration or cardiac condition characterization and abnormality detection, comprising:
an interface for receiving data representing a signal indicating concentration of carbon dioxide in patient gases over a plurality of signal cycles;
a signal processor for using the received data in determining at least one frequency related characteristic value comprising at least one of,
(a) a dominant frequency of said signal and
(b) a value derived by integration of the square of frequency over a bandwidth of said signal and comparing the frequency related characteristic value with a corresponding normal derived value; and
a patient monitor for generating an alert message associated with the comparison, in response to the comparison.

16. A system according to claim 15, wherein
said signal processor generates a ratio of said frequency related characteristic value to said corresponding normal derived value to compare values and
said patient monitor generates said alert message in response to the ratio exceeding a predetermined threshold.

17. A system according to claim 16, wherein
said corresponding normal derived value is at least one of, (a) a corresponding normal value for the patient concerned determined on a previous occasion and (b) a corresponding normal value for a population of patients having similar demographic characteristics as the patient.

18. A system for respiration or cardiac condition characterization and abnormality detection, comprising:
an interface for receiving sampled data representing a signal indicating concentration of carbon dioxide in patient gases over a plurality of cardiac cycles;
a signal processor for using the received sampled data in determining a mutual correspondence measure of the signal and a corresponding signal determined for the patient on a previous occasion; and
a patient monitor for generating an alert message associated with the comparison, in response to the comparison.

19. A system according to claim 18, wherein said mutual correspondence measure is derived using a function of the form,
$$MC ( {S}_{1} , {S}_{2} ) = \frac{{\sum }_{i = 1}^{N} {p}_{i} ( {s}_{1 i} , {s}_{2 i} )}{N}$$
where pᵢ is the joint probability of signal values s₁ᵢ and s₂ᵢ at sample point i, and the probability is summed over N sample points and normalized by dividing the sum by the value N.
